(19) United States
(12) Patent Application Publication
(10) Pub. No.: US 2017/0186446 A1
(43) Pub. Date: Jun. 29, 2017

(54) MOUTH PROXIMITY DETECTION

(71) Applicants: Michal Wosk, Tel Aviv (IL); Oded Vainas, Petah Tiqwa (IL)

(72) Inventors: Michal Wosk, Tel Aviv (IL); Oded Vainas, Petah Tiqwa (IL)

(21) Appl. No.: 15/388,977

(22) Filed: Dec. 22, 2016

(30) Foreign Application Priority Data
Dec. 24, 2015 (US) PCT/US2015/000437

(51) Int. Cl. G10L 25/78; G01C 5/06; G01N 33/50; G06F 3/0346; G06F 3/16; G10L 15/25; G10L 15/22; G01C 19/00; G01N 33/497

(52) U.S. Cl. CPC G10L 25/78; G01C 19/00; G01C 5/06; G01N 33/50; G01N 33/497; G06F 3/167; G10L 15/25; G10L 15/22; G06F 3/0346; G10L 2015/223

(57) ABSTRACT

Systems, apparatuses and methods may provide for a mouth proximity detection system to be used with a device, such as a wearable device, to determine when to activate (and/or deactivate) a voice-activated circuit on the device. Embodiments may utilize three layers of analysis to make the determination, including a layer to analyze motion, a layer to detect a mouth of a user, and a layer to fuse the layers in a power saving arrangement to determine proximity of the device to the mouth of the user and to determine whether to activate a voice-activated circuit on the device.

### CROSS-REFERENCE RELATED APPLICATIONS

The present application claims benefit of priority to International Patent Application No. PCT/US2015/000437, filed Dec. 24, 2015.

### TECHNICAL FIELD

Embodiments generally relate to wearable devices that interact with a user's voice. More particularly, embodiments relate to devices in which a user's voice is used to control a circuit, such as a recorder or command issuer, based on proximity of a device to a mouth of a user.

### BACKGROUND

Wearable devices such as smart watches, smart bracelets, smart rings, etc., or hand-held devices such as computer tablets, computer notebooks, smart phones, etc., may include an interface with buttons to activate various features on the devices. Buttons may offer a reliable mechanism for triggering a circuit on a wearable device, but generally require a user pressing and/or touching one or more of the buttons with a free hand, which may be inconvenient in some contexts and dangerous in others (e.g., while driving, during a surgical procedure, etc.).

Another approach to controlling such devices entails speaking into a microphone of the device and using voice control. Circuits on such devices may be activated for a variety of purposes, such as to record the user's voice, to transmit a message, and so forth. In general, the task of processing speech so that it may be used to control a circuit is computationally demanding and/or taxing on available power resources. Moreover, the use of voice commands may subject the device to inadvertent activation through false-positive commands in which circuitry is activated against the intention of the user. False activations may occur, for example, when a microphone on the device picks up third party speech or responds to other inputs from the local environment that the user does not intend to trigger activation of the device. In addition, such unintended activations of a circuit may waste battery power on the device. Thus, existing interfaces may be inconvenient and/or impose substantial burdens on available power resources.

### BRIEF DESCRIPTION OF THE DRAWINGS

The various advantages of the embodiments will become apparent to one skilled in the art by reading the following specification and appended claims, and by referencing the following drawings, in which:

FIGS. 1A-1C are example depictions of a user wearing a wearable device according to an embodiment;

FIG. 2 is an example of a block diagram of a multi-layered approach to determine activation of a circuit according to an embodiment;

FIG. 3 is an example of a block diagram of a system to activate a circuit according to an embodiment;

FIG. 4 is a flowchart of an example of a method of activating a circuit according to an embodiment; and

FIG. 5 is a block diagram of an example of a computing system according to an embodiment.

### DESCRIPTION OF EMBODIMENTS

As used herein, the term “wearable device” (or simply a “wearable”) may include clothing and/or accessories that incorporate computer and/or other electronic technologies. Examples of a wearable device may also include apparatuses including electronic processors that are arranged to be worn by a person and that are integrated into a wearable structure such as a wristband, a glove, a ring, eyeglasses, a belt-clip or a belt, an arm-band, a shoe, a hat, a shirt, an undergarment, an outer garment, clothing generally, and/or fashion accessories such as wallets, purses, umbrellas, and so forth. In embodiments, a wearable device may be implemented to provide all or part of computing functionality such as a functional capability of a smart phone, a tablet computer, a gaming device capable of executing computer applications, voice communications, data communications, and so forth. For example, some embodiments disclosed herein are presented in the context of a smart watch.

The term “smart” may be used to describe a device, such as “smart watch” or “smart glasses”, “smart wrist band”, etc., that includes one or more capabilities associated with smart phones such as geo-location capability, an ability to communicate with another device, an interactive display, multi-sensing capabilities, and/or other feature. Thus, a wearable may be a smart device when the wearable has access to one or more of the capabilities included in a smart phone such as geo-location, sensors, access to the internet via Wi-Fi (Wireless Fidelity, e.g., Institute of Electrical and Electronics Engineers/IEEE 802.11-2007, Wireless Local Area Network/LAN Medium Access Control (MAC) and Physical Layer (PHY) Specifications), near field communications, Bluetooth (e.g., IEEE 802.15.1-2005, Wireless Personal Area Networks) or other communication protocol. The access to one or more capabilities may be direct access and/or may be indirect access such via a Bluetooth connection with a nearby smart phone, a wearable device worn elsewhere on the user's person, and so forth.

A wearable device may include an interface to interact with a user. For example, a user may interact with a wearable device via voice activation of a circuit and/or circuitry associated with the wearable device. A smart watch may include, for example, a microphone to pick up a user's voice and analyze the voice and/or other sound to activate circuitry such as a transmitter, a voice recorder, and so forth. Voice activation may offer advantages over manually pressing buttons since it may not require that a user press any buttons with a free hand. However, voice activation may constitute a power intensive activity relative to pushing a button. Also, extraneous sounds in the environment that may be picked up by a microphone on the wearable device may cause the wearable device to activate a circuit such as a transmitter, a voice recorder, etc., when the user does not intend for the circuit to be activated. Thus, on-board power resources may be wasted. Embodiments disclosed herein may minimize unintended activations.

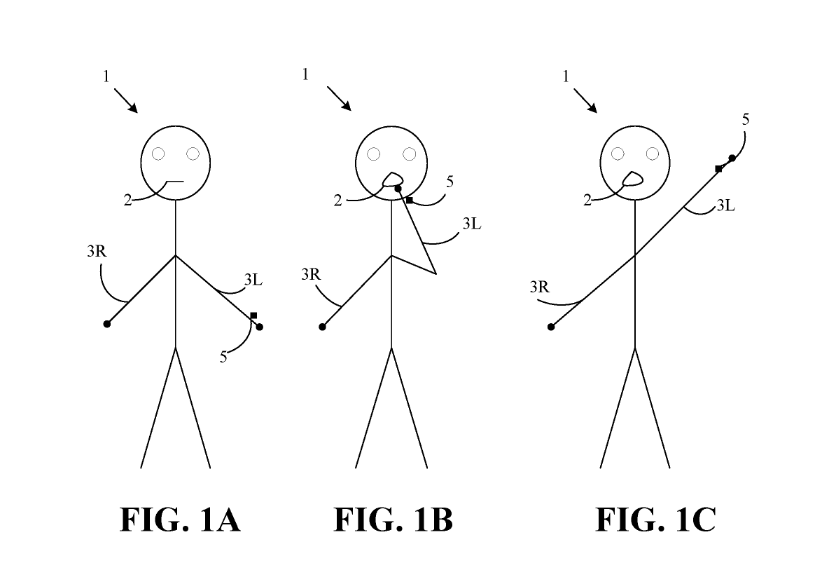

Turning now to FIG. 1A, a user 1 is depicted in a first position in which a mouth 2 of the user 1 is closed, and in which a right arm 3R and a left arm 3L are in a generally lowered position. The user 1 is wearing a smart watch 5 on a wrist of the left arm 3L, and in the illustrated position, the smart watch 5 is not proximate to the mouth 2 of the user 1. Any or all of these aspects of user position relative to the smart watch 5, including the closed mouth and/or the lowered arm, strongly suggest that the user 2 has no intention of speaking into the smart watch 5 to issue a spoken command, and/or that the user 2 is otherwise attempting to use his voice to engage the smart watch 5.

Referring to FIG. 1B, however, the user 2 has raised the left arm 3L so that the smart watch 5 is proximate to mouth 5, which is now open. The raised position of left arm 3L, the proximity of the smart watch 5 to the mouth 2 of the user 1, and the state of the mouth 2 of the user 1 (e.g., open), strongly suggest that the user is attempting to use voice commands to engage one or more features on the smart watch 5. Referring to FIG. 1C, the mouth 2 of the user 1 remains open and the user may be speaking, and the left arm 3L is fully extended away from the mouth 2 (e.g., generally away from the user's face). Although an open mouth may suggest speech, which may positively correlate with an attempt to issue one or more vocal commands, the position of the smart watch 5 away from the mouth 2 of the user 1 may suggest that perhaps the user 1 is engaged in some other activity, and that his speech may not be directed to the smart watch 5 (e.g., instead directed to someone nearby). Therefore, the user 1 in FIG. 1C may not be attempting to use voice commands to engage one or more features on the smart watch 5.

Embodiments provide a multi-layered approach of modeling various situations to efficiently and accurately determine when a user is acting with the intent of activating a wearable device through voice command and/or when the user is acting without such intent. Data from multiple sources may be considered and combined at multiple layers of analysis to provide an efficient and accurate way of determining when a user is attempting to use his voice to engage a feature on a wearable device such as, for example, a smart watch.

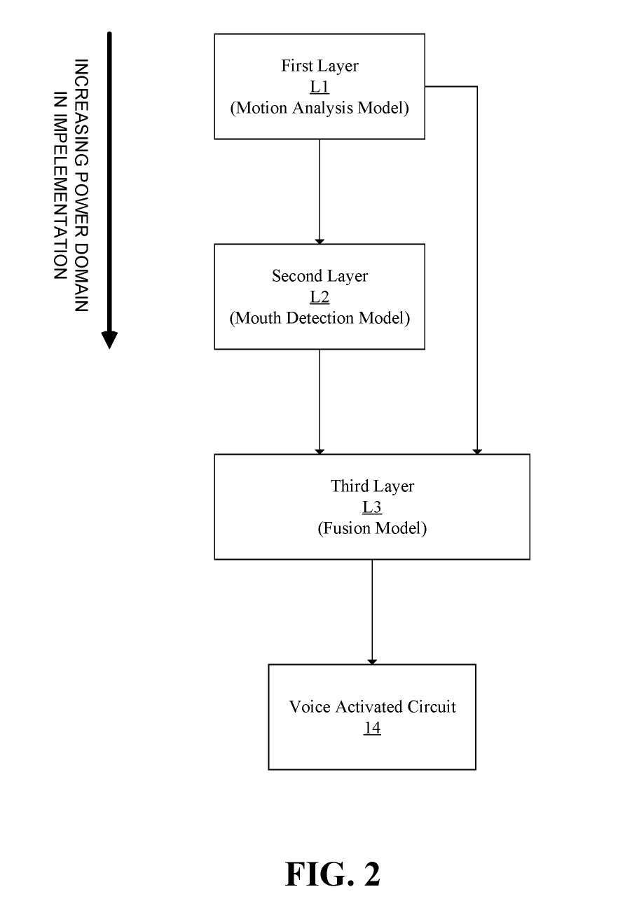

FIG. 2 is a block diagram illustrating employing three layers of analysis, L1, L2, and L3, according to an embodiment. Each of the layers may correspond to a model or set of models tasked with evaluating a given set of data. The first encountered the layer, L1, may, in some embodiments, run in the background on the device whenever the device is powered on. Layer L1 may examine data indicative of wearable device acceleration, position and/or orientation to determine a probability that the user may be attempting to use voice control over a device, such as the smart watch 5 (FIGS. 1A-1C.), discussed above.

For example, if a device is determined to be in motion towards a user's mouth, and if the device is oriented in a position in which a user might plausibly speak into a microphone on the device, then a motion analysis model used in layer L1 may determine that the user intends to vocally interact with the device. The motion analysis model may include, for example, an algorithmic component to identify whether movement of a wearable is towards or is away from the user's mouth. The algorithmic component may include multiple sub-models, each of which may identify a different movement such as, e.g., hand raising as show in FIG. 1C, activity detection (e.g. a sport), user gesture, user gait, etc., to make a determination that the movement detected is suggests voice activation.

Layer L1 may make the determination in terms of probabilities that are weighed against a movement threshold. The movement threshold may be a characteristic of layer L1 that is satisfied before the layer L1 determines that a movement of a device indicates and/or suggests that a user intends voice activation of the device. The absolute value of the movement threshold may depend, in some cases, on acceptable rates of false positives (e.g., a user seems to be seeking to use voice activation, but really is not) versus acceptable rates of false negatives (e.g., a user seems not to be trying to use voice activation, but really is) for a given context.

When the movement threshold is satisfied, then the first layer L1 invokes a second layer L2, which may use a mouth detection model to determine a probability that the device is physically near the user's mouth. The layer L2 may examine data indicative of the presence of a user's breath, wherein the breath may suggest proximity of the device to a user's mouth. The layer L2 may also examine data indicative of voice detection. In addition, the layer L2 may indicate a nearby presence of the user's mouth.

The layer L2 may make the determination in terms of probabilities that are judged against a mouth detection threshold. The mouth detection threshold may be a probability characteristic of the layer L2 that is satisfied before the layer L2 determines that a mouth has been detected. The absolute value of the mouth detection threshold may vary based on acceptable rates of false positives (e.g., a user seems to be seeking to use voice activation, but really is not) versus false negatives (e.g., a user seems not to be trying to use voice activation, but really is).

When the mouth detection threshold is satisfied, then the third layer L3 is invoked. Data from the first layer L1 and from the second layer L2 may be passed to the third layer L3, wherein a fusion model may weigh the data provided and/or the analysis generated by the first two layers L1 and L2. The fusion model of layer L3 may make a final determination of a probability that the user's mouth has been detected and that the user intends to use voice activation to control a voice activated circuit on the device. Layer L3 may make the determination with respect to a probability threshold characteristic of layer L3. When the probability threshold is satisfied, the layer L3 activates a voice-activated circuit (e.g., a recorder, a transmitter, etc.) on the device.

Thus, three layers of analysis, layer L1, layer L2, and layer L3, are serially and selectively engaged in a staged manner before a voice-activated circuit may be activated. As discussed below, the first layer L1 and the second layer L2 may be arranged in order of increasing power usage, both in terms of the computational resources and the electrical power they may require. Thus, layer L2 is at a relatively higher power domain with respect to layer L1. Also, the voice-activated circuit itself may belong to yet a higher level power domain. For example, if the voice-activated circuit is a transmitter and/or a recorder, the voice-activated circuit may require relatively more power than any of the three layers L1, L2, L3 engaged in determining whether to activate the voice-activated circuit.

In embodiments, layers belonging to relatively higher power domains may not be invoked unless first triggered by a result provided by a previous (and relatively lower power domain) layer. For example, the second layer L2 may be invoked only when the first layer L1 has satisfied a movement threshold. In another example, the third layer L3 may be invoked only when the second layer L2 has satisfied a mouth detection threshold. In a further example, the voice-activated circuit may be powered on only when a third layer L3 probability threshold has been satisfied. Thus, the arrangement may provide for voice activation of a voice-activated circuit, which is a relatively convenient and/or ergonomically friendly interface, while reducing a frequency of false indications that a user has issued a voice command. By reducing false indications, power resources may be saved. The arrangement may also serve to switch off a voice-activated circuit that is already on if any layer fails to meet its threshold, also conserving available power resources.

In embodiments a calibration step may be performed prior to engaging the layers L1, L2, and L3, in which the aforementioned models and thresholds may be selected based on a particular user's characteristics. The user characteristics may include the shape of the user's mouth, the characteristics of the user's voice, user arm length, user height, user gait characteristics, etc.

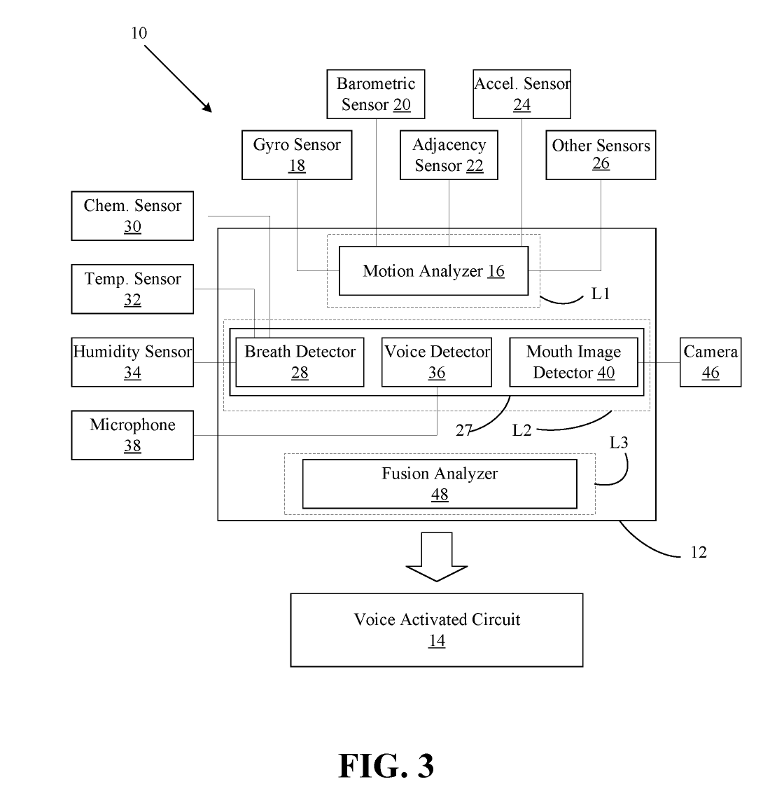

FIG. 3 is a block diagram of an embodiment of a mouth proximity detection system 10 in which a mouth proximity detector 12 determines whether a voice-activated circuit 14 on a device is to be activated. In the interest of economy of description, embodiments discussed herein are presented in terms of a voice-activated circuit 14 that is part of a wearable device. The wearable device may be a smart watch, a smart bracelet, a smart ring, and/or other wearable article incorporating electronics. More generally, the voice-activated circuit may be part of a computer tablet, computer, laptop, smart phone, and/or other mobile electronic device that may be worn and/or held by a user.

The first layer L1, shown in broken line in FIG. 3, may be implemented by a motion analyzer 16 that receives data concerning motion and position from a plurality of sensors that may be located on, inside of, and/or in close proximity to the wearable. In the illustrated example, the motion analyzer 16 receives data from a gyroscopic sensor 18, a barometric sensor 20, an adjacency sensor 22, an accelerometer sensor 24, and other sensors 26 that may be useful to measure position, displacement, velocity, and/or acceleration of the wearable.

The gyroscopic sensor 18 detects changes in the orientation of a wearable. The orientation of a wearable may provide important clues as to how a user may intend to use the wearable. Some orientations, such as those in which a microphone on a wearable device is oriented to face of a user, may more strongly suggest that the user is or is about to speak into the microphone than do other orientations, such as an orientation in which the microphone faces away from the user's mouth. In addition, the motion analyzer 16 may account for orientation and changes in orientation to determine a probability that a user is or shortly intends to speak into the wearable to activate a circuit, such as the voice activated circuit 14.

The barometric sensor 20 detects air pressure, which indicates altitude and changes in altitude of the wearable. A wearable, such as a smart watch worn on a wrist, which is raised to the altitude of a user's mouth (where the user is sitting or standing) will experience a local decline in air pressure. Values of air pressure and changes in levels of air pressure may be measured by the barometric sensor 20 or through an analysis of data provided by the barometric sensor 20. Data that indicates a decline in air pressure, as measured at the barometric sensor 20, may suggest that the wearable device is being raised towards the user's mouth as shown in FIG. 1B, which may suggest to the motion analyzer 16 that the user intends to or is speaking into the wearable.

Barometric data that corresponds to a stance, such as the stance shown in FIG. 1A, in which the wearable is well below the user's mouth, may suggest that the user does not intend to engage a voice activated circuit on the smart watch. Alternatively, if a user's hand is at too high an elevation with respect to the user's face, as shown in FIG. 1C, may suggest that the user may be raising an arm to engage in some other sort of activity rather than seeking to engage with a wearable device on a wrist. Thus, the motion analyzer 16 may use barometric data to make a determination of a probability that a user is attempting to issue a voice command to a wearable.

The adjacency sensor 22 may take advantage of other technologies that indicate the nearness of the wearable to the user's face and mouth. For example, if a user is wearing an earpiece or other head-based wearable device having a circuit that is capable of emitting and/or receiving a near-field signal or an infra-red (IR) signal, then a complementary circuit in the wearable may be able to determine distance and/or position with respect to the user's mouth based on the signal. In response, the motion analyzer 16 may use the data from the adjacency sensor 22 to determine a probability that the user is or shortly intends to begin voice control over the wearable.

The accelerometer sensor 24 measures local acceleration and may also provide indication of local gravity. Data provided by the accelerometer sensor 24 may be processed to further indicate whether the user is moving the wearable towards his mouth. The data may suggest that the user is about to or is speaking into the wearable. In addition, other sensors 26 may be provided as may exist or be developed to provide indication of the nearness of a wearable device to a user's mouth, using indications of elevation based on telemetry, global positioning system (GPS) data, etc.

The motion analyzer 16 weighs the data provided by sensors 18-26 to determine a probability that the wearable device is being moved towards the user's mouth or that it may be near the user's mouth. In this case, activation of a voice-activated circuit may be initiated. Conversely, the motion analyzer 16 may determine that the wearable device is being moved away from the user's mouth, in which case deactivation of the voice-activated circuit may be suggested. Models may be implemented in different ways. For example, in one embodiment, numerical values may be associated with the data provided by each of the sensors 18-26, which may be linearly summed together with suitable weighting functions for each type of data to determine a probability value. If the probability value is less than a movement threshold value of the motion analyzer 16 (which may be identical to the layer L1 movement threshold value, discussed above), then the voice-activated circuit 14 is not activated. In addition, if the voice-activated circuit 14 is already is in an activated state, the voice-activated circuit 14 may be deactivated.

On the other hand, if the probability value generated by the motion analyzer 16 equals or exceeds the movement threshold of the motion analyzer 16, then a mouth detector 27 (implementing the mouth detection layer L2 shown in broken line in FIG. 3) may be invoked. The mouth detector 27 includes a breath detector 28, a voice detector 36, and a mouth image detector 40, each of which may use sensor data to determine if the wearable device is proximate to the user's mouth.

A chemical sensor 30 detects chemical components of human breath and provides indication of human breath to the breath detector 28. Human breath may provide a chemical signature distinct from the general environment, and detection of breath may indicate that the wearable is proximate to the user's mouth. A user's breath may also be characterized by temperature and humidity. Accordingly, a temperature sensor 32 provides a measure of temperature and a humidity sensor 34 provides a measure of humidity in the area proximate to the wearable. Collectively, data provided by the sensors 30-32 may be used by the breath detector 28 to determine the presence of human breath in the immediate vicinity of the wearable. Detection of breath may be a strong marker that the wearable is proximate the user's mouth, and that the user is speaking into the wearable to activate a voice activated circuit.

The voice detector 36 uses data provided by, for example, a microphone 38, to detect a user's voice. The level of analysis performed by the voice detector may vary, ranging from basic detection of sounds corresponding to a human voice, to identification of a specific user's voice. Detection of a voice may be a strong marker that the wearable is proximate to the user's mouth, and that the user is speaking into the wearable to activate it's voice activated circuit.

Additionally, a camera 46 may capture an image to be analyzed at a mouth image detector 40. Identification the user's mouth being proximate to the wearable may suggest that the user has placed the wearable near his mouth and is or is attempting to use his voice to control a voice activated circuit on the wearable.

The mouth detector 27 weighs the data provided by sensors 30-38 and 46 to determine a probability that the user's mouth has been detected near the wearable. In this case, activation of a voice-activated circuit may be called for, subject to further analysis by the fusion analyzer 48 discussed below. Conversely, the mouth detector 27 may weigh the data provided by the sensors 30-38 and 46 to determine a probability that the user's mount has not been detected near the wearable. In this case, the wearable is not near the user's mouth, and a voice-activated circuit may be deactivated. The model may be implemented in different ways. For example, in one embodiment, numerical values may be associated with the data provided by each of the sensors 30-38 and 46, which may be linearly summed together with suitable weighting functions for each type of data to determine a probability value. If the probability value is less than a specified threshold value (which may be the mouth detection threshold of layer L2), then the voice-activated circuit 14 is not activated. In addition, if the voice-activated circuit 14 is already is in an activated state, the voice-activated circuit 14 may be deactivated.

On the other hand, if the probability value generated by the mouth detector 27 equals or exceeds the mouth detection threshold, then the fusion analyzer 48 (corresponding to the third layer L3 shown in broken line in FIG. 3) may be invoked. The fusion analyzer 48 considers inputs from the motion analyzer 16 and the mouth detector 27 including, in some embodiments, outputs provided by the breath detector 28, the voice detector 36, and the mouth image detector 40, and applies heuristics to determine a probability that a user has placed the wearable near his mouth and is attempting to exercise voice activated control over a voice activated circuit on the wearable. If the fusion analyzer 48 determines that a probability threshold has been satisfied, then the fusion analyzer 48 activates the voice-activated circuit 14. If, however, the probability threshold has not been satisfied, then the voice-activated circuit is not activated or, conversely if already activated, may be powered off.

In general, the voice-activated circuit (e.g., a transmitter) may be at a relatively higher power domain than the fusion analyzer 48, and the mouth detector may be at a relatively higher power domain than the motion analyzer 16. Embodiments may not trigger a higher power domain until it is warranted by a determination made at a lower power domain, thereby saving power.

The motion analyzer 16 may be operated at a higher frequency than the mouth detector 27 or the fusion analyzer 48, since the motion analyzer 16 may typically operate before either the mouth detector 27 or the fusion analyzer 48 may be engaged. In one embodiment, the motion analyzer 16 may be kept operating in the background whenever the wearable is powered on. In other embodiments, the motion analyzer 16 may be triggered to an “on” state whenever any threshold level of input is received from one or more of the sensors 18-26.

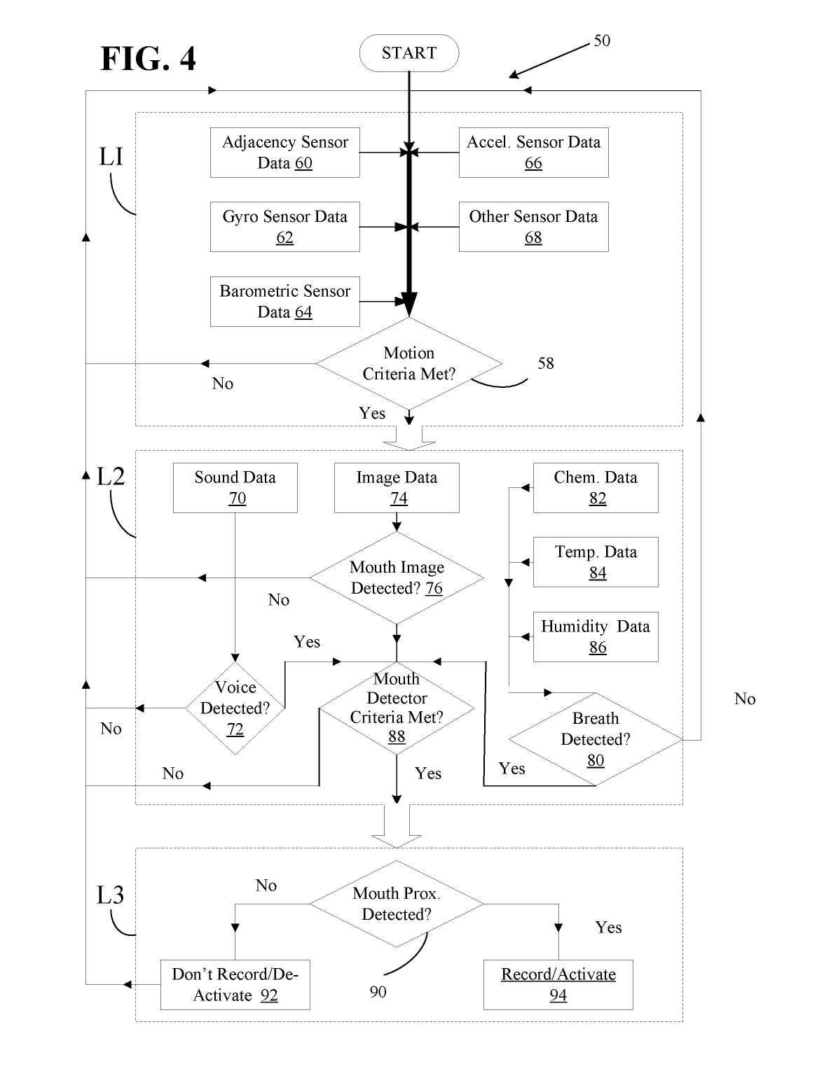

FIG. 4 shows a flowchart of an example of a method 50 of detecting the proximity of a user's mouth to a wearable device that may include a voice recording circuit as a voice-activated circuit. The method 50 may be implemented as one or more modules in a set of logic instructions stored in a machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof.

The method 50 may be implemented by a first layer L1, a second layer L2, and a third layer L3, which may be same as the layers L1-L2 (FIGS. 2-3), discussed above. Each of the layers L1-L3 is shown in broken line in the method 50.

The first layer L1 has a block 58 that determines whether motion criteria have been satisfied based on adjacency sensor data 60, gyroscopic sensor data 62, barometric sensor data 64, accelerometer sensor data 66, and/or other sensor data 68. The motion criteria may include detection of a movement and/or motion of the wearable in a direction towards a mouth of a user, and may be based on multiple models, including a model of user gait, a model of specific movements (such as raising or lowering an arm), a model of user gesture, a model of user or wearable tilt, etc. The motion criteria may include one or more threshold values indicative of gait, gesture, tilt, etc. If the block 58 determines that the criteria have not been met, then control returns to the start, and the method awaits new sensor inputs.

If the block 58 determines that the criteria have been met, then the second layer L2 is invoked to determine if a mouth has been detected. Illustrated processing block 70 passes sound data, for example provided via the microphone 38 (FIG. 3), discussed above, to block 72, which determines if a voice detection threshold indicative of voice detection has been satisfied. If the voice detection threshold has not been satisfied, then control loops back to the start of the layer L1. In other embodiments, control may continue to block 88 instead of flowing back to the start of the layer L1.

Illustrated processing block 74 passes image data, for example provided via a camera such as the camera 46 (FIG. 3), discussed above, to block 76, which determines if a mouth image detection threshold indicative of mouth image detection has been satisfied. If the mouth image threshold has not been satisfied, then control loops back to the start of the first layer L1. In other embodiments, control may continue on to processing block 88 instead of flowing back to the start of the layer L1.

Illustrated block 80 detects the presence of breath based on chemical data provided by processing block 82, temperature data provided by processing block 84, and humidity data provided by processing block 86. The block 80 determines if a breath detection threshold indicative of the presence of breath has been satisfied. If the breath detection threshold has not been satisfied, then control loops back to the start of the layer L1. In other embodiments, control may continue to block 88 instead of flowing back to the start of the layer L1.

If any or all of the decisions made by the block 72, the block 76, or the block 80 are YES, then block 88 determines if the mouth detector criteria have been met. This determination may be made based on weighted determinations provided by, for example, the breath detector 28, the voice detector 36, and/or the mouth image detector 40 (FIG. 3), discussed above. Also, the determination may be balanced against weighted threshold values for each. The criteria may include meeting the second layer L2 mouth detector threshold, discussed above. In some embodiments, block 88 may directly determine whether the mouth detector criteria have been satisfied by using any or all of the sound data from block 70, the image data from block 74, the chemical data from block 82, the temperature data from block 84, and humidity data from block 86. If block 88 determines that the mouth detector criteria have not been satisfied, then control passes back to the start of layer L1.

If, on the other hand, block 88 determines that the criteria have been satisfied, then the third layer L3 is invoked to make a final determination at block 90 of whether a mouth has been detected proximal to the wearable. The decision may be based on any or all of the weighted outputs of the previous layers L1 and L2, as well as other heuristics reflective of user behavior. If the final determination is NO, then the voice-activated circuit (in this example, to control a voice recorder) is not activated or, if it is already on, it is deactivated at processing block 92. On the other hand, if the final determination at block 90 is YES, then the voice-activated circuit is activated at processing block 94, and voice recording (or other voice activated feature) is turned on.

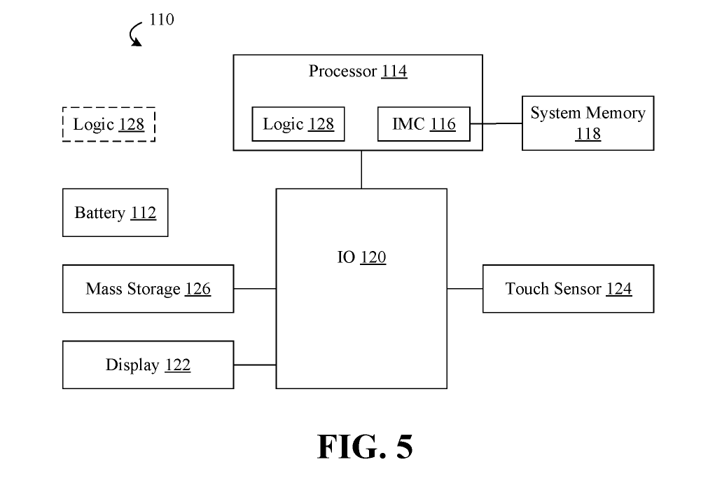

Turning now to FIG. 5, a computing device 110 is illustrated according to an embodiment. The computing device 110 may be part of a platform having computing functionality (e.g., personal digital assistant/PDA, notebook computer, tablet computer), communications functionality (e.g., wireless smart phone), imaging functionality, media playing functionality (e.g., smart television/TV), wearable functionality (e.g., watch, eyewear, headwear, footwear, jewelry) or any combination thereof (e.g., mobile Internet device/MID). In the illustrated example, the device 110 includes a battery 112 to supply power to the device 110 and a processor 114 having an integrated memory controller (IMC) 116, which may communicate with system memory 118. The system memory 118 may include, for example, dynamic random access memory (DRAM) configured as one or more memory modules such as, for example, dual inline memory modules (DIMMs), small outline DIMMs (SODIMMs), etc.

The illustrated device 110 also includes a input output (TO) module 120, sometimes referred to as a Southbridge of a chipset, that functions as a host device and may communicate with, for example, a display 122 (e.g., touch screen, liquid crystal display/LCD, light emitting diode/LED display), a touch sensor 124 (e.g., a touch pad, etc.), and mass storage 126 (e.g., hard disk drive/HDD, optical disk, flash memory, etc.). The illustrated processor 114 may execute logic 128 (e.g., logic instructions, configurable logic, fixed-functionality logic hardware, etc., or any combination thereof) configured to function similarly to the system 10 (FIG. 3). Thus, the computing device 110 may provide mouth detection that may be used to trigger a voice activated circuit.

### Additional Notes and Examples

Example 1 may include an apparatus to control a circuit based on proximity of a device to a mouth of a user, comprising a motion analyzer to detect movement of a device towards a mouth of a user based on first sensor data, a mouth detector to detect the mouth of the user based on second sensor data, and a fusion analyzer to determine a probability that the device is in proximity to the mouth of the user in response to receiving output from the motion analyzer and the mouth detector, and activate a circuit in response to the probability satisfying a probability threshold.

Example 2 may include the apparatus of Example 1, wherein the motion analyzer is to invoke the mouth detector to detect the mouth of the user only in response to a movement threshold being satisfied, and wherein the mouth detector is to invoke the fusion analyzer to determine the probability only in response to a mouth detection threshold being satisfied.

Example 3 may include the apparatus of any one of Examples 1 to 2, wherein the mouth detector is to detect the mouth of the user at a higher power domain relative to the motion analyzer that is to detect movement of the device towards the mouth of the user.

Example 4 may include the apparatus of any one of Examples 1 to 3, wherein the mouth detector includes a breath detector to detect a presence of breath, a voice detector to detect a voice, and an image detector to detect an image of a mouth.

Example 5 may include the apparatus of any one of Examples 1 to 4, further including one or more of a gyroscopic sensor, a barometric sensor, a proximity sensor, or an accelerometer to generate the first sensor data, and one or more of a chemical sensor, a temperature sensor, or a humidity sensor to generate the second sensor data.

Example 6 may include the apparatus of any one of Examples 1 to 5, wherein the circuit is to include a voice activated circuit, and wherein the probability includes a determination that the user is to be presently speaking.

Example 7 may include a device to control a circuit based on proximity of a device to a mouth of a user, comprising a motion analyzer to detect movement of a device towards a mouth of a user based on first sensor data, a mouth detector to detect the mouth of the user based on second sensor data, a fusion analyzer to determine a probability that the device is in proximity to the mouth of the user based on output from the motion analyzer and the mouth detector, and a circuit to be activated by the fusion analyzer at least in response to the probability satisfying a probability threshold.

Example 8 may include the device of Example 7, wherein the motion analyzer is to invoke the mouth detector to detect the mouth of the user when in response to a movement threshold being satisfied, and wherein the mouth detector is to invoke the fusion analyzer to determine the probability only in response to a mouth detection threshold being satisfied.

Example 9 may include the device of any one of Examples 7 to 8, wherein the mouth detector is to detect the mouth of the user at a higher power domain relative to the motion analyzer that is to detect movement of the device towards the mouth of the user.

Example 10 may include the device of any one of Examples 7 to 9, wherein the mouth detector includes a breath detector to detect a presence of breath, a voice detector to detect a voice, and an image detector to detect an image of a mouth.

Example 11 may include the device of any one of Examples 7 to 10, further including one or more of a gyroscopic sensor, a barometric sensor, a proximity sensor, or an accelerometer to generate the first sensor data, and one or more of a chemical sensor, a temperature sensor, or a humidity sensor to generate the second sensor data.

Example 12 may include the device of any one of Examples 7 to 11, wherein the circuit is to include a voice activated circuit, and wherein the probability includes a determination that the user is to be presently speaking.

Example 13 may include the device of any one of Examples 7 to 12, wherein the device is to be wearable on one or more of an arm, a wrist, a hand, a finger, a toe, a foot, a leg, a torso, an article of clothing, or a fashion accessory of the user.

Example 14 may include at least one computer readable storage medium comprising a set of instructions, which when executed by an apparatus, cause the apparatus to detect movement of a device towards a mouth of a user based on first sensor data, detect the mouth of the user based on second sensor data, determine a probability that the device is in proximity to the mouth of the user based on a detection of movement of the device towards the mouth and a detection of the mouth, and activate a circuit of a wearable device at least in response to the probability satisfying a probability threshold.

Example 15 may include the at least one computer readable storage medium of Example 14, wherein the instructions, when executed, cause the apparatus to detect the mouth of the user only in response to a movement threshold being satisfied, and determine the probability only in response to a mouth detection threshold being satisfied.

Example 16 may include the at least one computer readable storage medium of any one of Examples 14 to 15, wherein the instructions, when executed, cause the apparatus to detect the mouth of the user at a higher power domain relative to detecting movement of the device towards the mouth of the user.

Example 17 may include the at least one computer readable storage medium of any one of Examples 14 to 16, wherein the instructions, when executed, cause the apparatus to detect a presence of breath, detect a voice, and detect an image of a mouth.

Example 18 may include the at least one computer readable storage medium of any one of Examples 14 to 17, wherein the instructions, when executed, cause the apparatus to generate the first sensor data by one or more of a gyroscopic sensor, a barometric sensor, a proximity sensor, or an accelerometer, and generate the second sensor data by one or more of a chemical sensor, a temperature sensor, or a humidity sensor.

Example 19 may include the at least one computer readable storage medium of any one of Examples 14 to 18, wherein the circuit includes a voice activated circuit, and wherein the probability includes a determination that a user is presently speaking.

Example 20 may include the at least one computer readable storage medium of any one of Examples 14 to 19, wherein the device is to be wearable on one or more of an arm, a wrist, a hand, a finger, a toe, a foot, a leg, a torso, an article of clothing, or a fashion accessory of the user.

Example 21 may include a method to control a circuit based on proximity of a device to a mouth of a user, comprising detecting movement of a device towards a mouth of a user based on first sensor data, detecting the mouth of the user based on second sensor data, determining a probability that the device is in proximity to the mouth of the user based on a detection of movement of the device towards the mouth and a detection of the mouth, and activating a circuit of a wearable device at least in response to the probability satisfying a probability threshold.

Example 22 may include the method of Example 21, further including detecting the mouth of the user only in response to a movement threshold being satisfied, and determining the probability only in response to a mouth detection threshold being satisfied.

Example 23 may include the method of any one of Examples 21 to 22, further including detecting the mouth of the user at a higher power domain relative to detecting movement of the device towards the mouth of the user.

Example 24 may include the method of any one of Examples 21 to 23, wherein detecting the mouth of the user further includes detecting a presence of breath, detecting a voice, and detecting an image of a mouth.

Example 25 may include the method of any one of Examples 21 to 24, further including generating the first sensor data by one or more of a gyroscopic sensor, a barometric sensor, a proximity sensor, or an accelerometer, and generating the second sensor data by one or more of a chemical sensor, a temperature sensor, or a humidity sensor.

Example 26 may include the method of any one of Examples 21 to 25, wherein the circuit includes a voice activated circuit, and wherein the probability includes a determination that a user is presently speaking.

Example 27 may include the method of any one of Examples 21 to 26, wherein the device is to be wearable on one or more of an arm, a wrist, a hand, a finger, a toe, a foot, a leg, a torso, an article of clothing, or a fashion accessory of the user.

Example 28 may include an apparatus to control a circuit based on proximity of a device to a user's mouth, comprising first means to detect movement of a device towards a mouth of a user based on first sensor data, second means for detecting the mouth of a user based on second sensor data, third means for determining a probability that the device is in proximity to the mouth of a user based on an output from the first means and an output from the second means, and means for activating a circuit at least when the probability is to satisfy a probability threshold.

Example 29 may include the apparatus of Example 28, wherein the first means is to invoke the second means only when a first means threshold is to be satisfied, and wherein the second means is to invoke the third means only when a second means proximity threshold is to be satisfied.

Example 30 may include the apparatus of any one of Examples 28 to 29, wherein the second means is at a higher power domain relative to the first means.

Example 31 may include the apparatus of any one of Examples 28 to 30, wherein the second means includes means for detecting a presence of breath, means for detecting a voice, and means for detecting an image of a mouth.

Example 32 may include the apparatus of any one of Examples 28 to 31, further including one or more of a gyroscopic sensor, a barometric sensor, a proximity sensor, or an accelerometer to generate the first sensor data, and one or more of a chemical sensor, a temperature sensor, or a humidity sensor to generate the second sensor data.

Example 33 may include the apparatus of any one of Examples 28 to 32, wherein the circuit is to include a voice activated circuit, and the probability includes a determination that the user is presently speaking.

Example 34 may include the apparatus of any one of Examples 28 to 33, wherein the device is to be wearable on one or more of an arm, a wrist, a hand, a finger, a toe, a foot, a leg, a torso, an article of clothing, or a fashion accessory of the user.

Example 35 may include a system to determine proximity of a device to a speaking user's mouth, comprising a device, a motion analyzer to detect movement of the device towards a mouth of a user based on first sensor data, a mouth detector to detect the mouth of the user based on second sensor data, a fusion analyzer to determine a likelihood that the device is in proximity to the mouth of the user and that the user is speaking into the device based on output from the motion analyzer and the mouth detector, wherein the fusion analyzer is to activate a circuit if the likelihood is greater than a threshold.

Example 36 may include the system of Example 35, wherein the circuit is a voice-activated circuit.

Example 37 may include the system of any one of Examples 35 to 36, wherein the circuit includes a voice recorder.

Example 38 may include the system of any one of Examples 35 to 37, wherein the circuit is a control circuit.

Example 39 may include the system of any one of Examples 35 to 38, wherein the circuit includes a microphone.

Example 40 may include the system of any one of Examples 35 to 39, wherein the device is a wearable device.

Example 41 may include the system of any one of Examples 35 to 40, wherein the device is one or more of a watch, a ring, or a bracelet.

Example 42 may include the system of any one of Examples 35 to 41, wherein the circuit is to be deactivated if the device is not proximal to a speaking user's mouth.

Example 43 may include a method to control a circuit based on proximity of a device to a mouth of a user, comprising calibrating one or more models and/or model thresholds of device movement and/or mouth detection based on characteristics of a user, detecting movement of a device towards a mouth of a user based on first sensor data, detecting the mouth of the user based on second sensor data, determining a probability that the device is in proximity to the mouth of the user based on a detection of movement of the device towards the mouth and a detection of the mouth, and activating a circuit of a wearable device at least when the probability satisfies a probability threshold.

Example 44 may include the method of Example 43, wherein the characteristics of a user may include one or more of the shape of the user's mouth, the characteristics of the user's voice, the user's arm length, the user's height, or the user's gait characteristics.

Embodiments are applicable for use with all types of semiconductor integrated circuit (“IC”) chips. Examples of these IC chips include but are not limited to processors, controllers, chipset components, programmable logic arrays (PLAs), memory chips, network chips, systems on chip (SoCs), SSD/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however, should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g., photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the platform within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections. In addition, the terms “first”, “second”, etc. may be used herein only to facilitate discussion, and carry no particular temporal or chronological significance unless otherwise indicated.

As used in this application and in the claims, a list of items joined by the term “one or more of” may mean any combination of the listed terms. For example, the phrases “one or more of A, B or C” may mean A, B, C; A and B; A and C; B and C; or A, B and C.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

We claim:

1. An apparatus, comprising:
a motion analyzer to detect movement of a device towards a mouth of a user based on first sensor data;
a mouth detector to detect the mouth of the user based on second sensor data; and
a fusion analyzer to
determine a probability that the device is in proximity to the mouth of the user in response to receiving output from the motion analyzer and the mouth detector, and
activate a circuit in response to the probability satisfying a probability threshold.

2. The apparatus of claim 1, wherein the motion analyzer is to invoke the mouth detector to detect the mouth of the user only in response to a movement threshold being satisfied, and wherein the mouth detector is to invoke the fusion analyzer to determine the probability only in response to a mouth detection threshold being satisfied.

3. The apparatus of claim 2, wherein the mouth detector is to detect the mouth of the user at a higher power domain relative to the motion analyzer that is to detect movement of the device towards the mouth of the user.

4. The apparatus of claim 1, wherein the mouth detector includes:
a breath detector to detect a presence of breath;
a voice detector to detect a voice; and
an image detector to detect an image of a mouth.

5. The apparatus of claim 4, further including:
one or more of a gyroscopic sensor, a barometric sensor, a proximity sensor, or an accelerometer to generate the first sensor data; and
one or more of a chemical sensor, a temperature sensor, or a humidity sensor to generate the second sensor data.

6. The apparatus of claim 1, wherein the circuit is to include a voice activated circuit, and wherein the probability includes a determination that the user is to be presently speaking.

7. A device, comprising:
a motion analyzer to detect movement of a device towards a mouth of a user based on first sensor data;
a mouth detector to detect the mouth of the user based on second sensor data;
a fusion analyzer to determine a probability that the device is in proximity to the mouth of the user based on output from the motion analyzer and the mouth detector; and
a circuit to be activated by the fusion analyzer at least in response to the probability satisfying a probability threshold.

8. The device of claim 7, wherein the motion analyzer is to invoke the mouth detector to detect the mouth of the user only in response to a movement threshold being satisfied, and wherein the mouth detector is to invoke the fusion analyzer to determine the probability only in response to a mouth detection threshold being satisfied.

9. The device of claim 8, wherein the mouth detector is to detect the mouth of the user at a higher power domain relative to the motion analyzer that is to detect movement of the device towards the mouth of the user.

10. The device of claim 7, wherein the mouth detector includes:
a breath detector to detect a presence of breath;
a voice detector to detect a voice; and
an image detector to detect an image of a mouth.

11. The device of claim 10, further including:
one or more of a gyroscopic sensor, a barometric sensor, a proximity sensor, or an accelerometer to generate the first sensor data; and
one or more of a chemical sensor, a temperature sensor, or a humidity sensor to generate the second sensor data.

12. The device of claim 7, wherein the circuit is to include a voice activated circuit, and wherein the probability includes a determination that the user is to be presently speaking.

13. The device of claim 7, wherein the device is to be wearable on one or more of an arm, a wrist, a hand, a finger, a toe, a foot, a leg, a torso, an article of clothing, or a fashion accessory of the user.

14. At least one computer readable storage medium comprising a set of instructions, which when executed by an apparatus, cause the apparatus to:
detect movement of a device towards a mouth of a user based on first sensor data;
detect the mouth of the user based on second sensor data;
determine a probability that the device is in proximity to the mouth of the user based on a detection of movement of the device towards the mouth and a detection of the mouth; and
activate a circuit of a wearable device at least in response to the probability satisfying a probability threshold.

15. The at least one computer readable storage medium of claim 14, wherein the instructions, when executed, cause the apparatus to:
detect the mouth of the user only in response to a movement threshold being satisfied; and
determine the probability only in response to a mouth detection threshold being satisfied.

16. The at least one computer readable storage medium of claim 15, wherein the instructions, when executed, cause the apparatus to detect the mouth of the user at a higher power domain relative to detecting movement of the device towards the mouth of the user.

17. The at least one computer readable storage medium of claim 14, wherein the instructions, when executed, cause the apparatus to:
detect a presence of breath;
detect a voice; and
detect an image of a mouth.

18. The at least one computer readable storage medium of claim 17, wherein the instructions, when executed, cause the apparatus to:
generate the first sensor data by one or more of a gyroscopic sensor, a barometric sensor, a proximity sensor, or an accelerometer; and
generate the second sensor data by one or more of a chemical sensor, a temperature sensor, or a humidity sensor.

19. The at least one computer readable storage medium of claim 14, wherein the circuit includes a voice activated circuit, and wherein the probability includes a determination that a user is presently speaking.

20. The at least one computer readable storage medium of claim 14, wherein the device is to be wearable on one or more of an arm, a wrist, a hand, a finger, a toe, a foot, a leg, a torso, an article of clothing, or a fashion accessory of the user.

21. A method, comprising:
detecting movement of a device towards a mouth of a user based on first sensor data;
detecting the mouth of the user based on second sensor data;
determining a probability that the device is in proximity to the mouth of the user based on a detection of movement of the device towards the mouth and a detection of the mouth; and
activating a circuit of a wearable device at least in response to the probability satisfying a probability threshold.

22. The method of claim 21, further including:
detecting the mouth of the user only in response to a movement threshold being satisfied;
determining the probability only in response to a mouth detection threshold being satisfied; and
detecting the mouth of the user at a higher power domain relative to detecting movement of the device towards the mouth of the user.

23. The method of claim 21, further including:
detecting a presence of breath;
detecting a voice; and
detecting an image of a mouth.

24. The method of claim 23, further including:
generating the first sensor data by one or more of a gyroscopic sensor, a barometric sensor, a proximity sensor, or an accelerometer; and
generating the second sensor data by one or more of a chemical sensor, a temperature sensor, or a humidity sensor.

25. The method of claim 21, wherein the circuit includes a voice activated circuit, the probability includes a determination that a user is presently speaking, and the device is to be wearable on one or more of an arm, a wrist, a hand, or a finger of the user.
